(19) United States
(12) Patent Application Publication
(10) Pub. No.: US 2021/0319869 A1
(43) Pub. Date: Oct. 14, 2021

(54) Methods and Systems for Interactive Children's Medication and Wellness Tracking

(71) Applicants: Meghana Kaipa, Pleasanton, CA (US); Siddharth Kaipa, Pleasanton, CA (US)

(72) Inventors: Meghana Kaipa, Pleasanton, CA (US); Siddharth Kaipa, Pleasanton, CA (US)

(21) Appl. No.: 17/114,187

(22) Filed: Dec. 7, 2020

Related U.S. Application Data
(60) Provisional application No. 62/944,795, filed on Dec. 6, 2019.

(51) Int. Cl. G16H 20/10; G16H 30/00; G06F 3/0481; A61J 7/04

(52) U.S. Cl. CPC G16H 20/10; G16H 30/00; A61J 2205/50; A61J 7/0409; A61J 2200/30; G06F 3/0481

(57) ABSTRACT

Interactive children's medication and wellness tracking system includes a mobile application having a processor, a camera, and a storage device. The storage device is commutatively coupled to the processor, and includes scanning logic to extract medication data from at least one picture or video stream captured by the camera. The system can also include a graphical display logic to receive child data associated with at least one child of a user. Communication logic can transmit the medication data to a children's medication server and subsequently receive dosage data in response to the transmitted medication data. Graphical display logic can further display the proper dosage of the scanned medicine wherein the dosage is based on at least the child data, and dosage data. The proper dosage may be generated within the mobile application or can be generated within the children's medication server and transmitted to the mobile application for display.

### PRIORITY

This application claims benefit of and priority to U.S. Provisional Application No. 62/944,795, filed Dec. 6, 2019, which is incorporated by reference in its entirety herein.

### FIELD OF INVENTION

The present disclosure technically relates to tracking and monitoring medication dispensing. More particularly, the present disclosure technically relates to dynamically tracking and providing dosing and safety information for children's medicine.

### BACKGROUND

Parents and caregivers are often faced with numerous challenges when trying to provide in-home medical care to their children and other patients. Children's smaller bodies leave less room for error when providing correct doses of over-the-counter medicine. Additionally, certain medical conditions can affect the correct doses needed to treat various conditions. Parents, often tired from dealing with sick children at odd hours of the day and night, often find it difficult to perform the correct calculations needed to create the proper dose of medicine needed. As a child's illness progresses and subsides, the dosage typically is reduced over time. Tracking this reduction can be difficult for parents. Additionally, as children age and grow quickly, dosages that were once correct for kids during a particular sickness, can often be incorrect later on when dealing with the same medicine. Although rare, parents must also be aware and up to date on any potential recalls of children medicine. As busy as parents usually are dealing with sick children, knowledge of recalls of children's medicine are often overlooked.

In certain cases, parents may feel overwhelmed in trying to determine a correct medicine or dosage for their child. Due to the often-long wait times to get into see a primary care doctor and the high costs of an emergency room visit during late hours, parents are often left with no guidance from medical professionals when dealing with their sick children. Furthermore, parents often lack unobtrusive feedback to their child's reaction to medication. Gathering vitals often requires a parent to wake their child up when they would rather the child continue to sleep and recover from their illness.

### BRIEF DESCRIPTION OF DRAWINGS

The above, and other, aspects, features, and advantages of several embodiments of the present disclosure will be more apparent from the following description as presented in conjunction with the following several figures of the drawings.

FIG. 1 is a system diagram of the interactive children's medication and wellness tracking system in accordance with an embodiment of the invention;

FIG. 2 is a schematic illustrating components of one or more interactive children's medication and wellness tracking computing devices in accordance with an embodiment of the invention;

FIG. 3 is a conceptual schematic illustrating various logics and data stores within an interactive children's medication and wellness tracking computing device in accordance with an embodiment of the invention;

FIG. 4A is a conceptual mock-up of an interactive children's medication and wellness tracking application in scan mode in accordance with an embodiment of the invention;

FIG. 4B is a conceptual mock-up of an interactive children's medication and wellness tracking application in dosage mode in accordance with an embodiment of the invention;

FIG. 4C is a conceptual mock-up of an interactive children's medication and wellness tracking application in call mode in accordance with an embodiment of the invention;

FIG. 4D is a conceptual mock-up of an interactive children's medication and wellness tracking application in account management mode in accordance with an embodiment of the invention; and

FIG. 5 is a flowchart depicting an interactive children's medication and wellness tracking process in accordance with an embodiment of the invention.

Corresponding reference characters indicate corresponding components throughout the several figures of the drawings. Elements in the several figures are illustrated for simplicity and clarity and have not necessarily been drawn to scale. For example, the dimensions of some of the elements in the figures might be emphasized relative to other elements for facilitating understanding of the various presently disclosed embodiments. In addition, common, but well-understood, elements that are useful or necessary in a commercially feasible embodiment are often not depicted in order to facilitate a less obstructed view of these various embodiments of the present disclosure.

### DETAILED DESCRIPTION

In response to the problems regarding providing effective children's medication and care, systems and methods are described herein that describe processes for interactive children's medication and wellness tracking. Specifically, in many embodiments, the interactive children's medication and wellness tracking can be accomplished through a mobile computing device such as, but not limited to, a cellular phone. In these embodiments, the user, typically a parent can provide basic vital info about a child (e.g., age, weight, allergies, etc.) and receive a proper and correct dosage from the interactive children's medication and wellness tracking application. In further embodiments, the interactive children's medication and wellness tracking application can also provide notification when additional doses are needed as well as providing dosing history, access to relevant medical info, and even provide audio/visual access to medical professionals to have questions answered without having to leave the house with the sick child.

Although various embodiments of the present disclosure discuss a parent and child relationship, those skilled in the art will recognize that the embodiments discussed herein can apply to non-children (such as those who have conditions that provide abnormal dosing levels, or adults with medical or physical conditions that require constant or near-constant care) and can be operated by any caregiver, medical professional, or parent. Any previous or subsequent discussion of parents or children is intended to include each of these types of user and/or patient.

The description herein is not to be taken in a limiting sense, but is made merely for the purpose of describing the general principles of exemplary embodiments. The scope of the disclosure should be determined with reference to the claims. Reference throughout this specification to “one embodiment,” “an embodiment,” or similar language means that a particular feature, structure, or characteristic that is described in connection with the referenced embodiment is included in at least the referenced embodiment. Likewise, reference throughout this specification to “some embodiments” or similar language means that particular features, structures, or characteristics that are described in connection with the referenced embodiments are included in at least the referenced embodiments. Thus, appearances of the phrases “in one embodiment,” “in an embodiment,” “in some embodiments,” and similar language throughout this specification can, but do not necessarily, all refer to the same embodiment.

Further, the described features, structures, or characteristics of the present disclosure can be combined in any suitable manner in one or more embodiments. In the description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure.

In the following description, certain terminology is used to describe features of the invention. For example, in certain situations, both terms “logic” and “engine” are representative of hardware, firmware and/or software that is configured to perform one or more functions. As hardware, logic (or engine) may include circuitry having data processing or storage functionality. Examples of such circuitry may include, but are not limited or restricted to a microprocessor, one or more processor cores, a programmable gate array, a microcontroller, a controller, an application specific integrated circuit, wireless receiver, transmitter and/or transceiver circuitry, semiconductor memory, or combinatorial logic.

Logic may be software in the form of one or more software modules, such as executable code in the form of an executable application, an application programming interface (API), a subroutine, a function, a procedure, an applet, a servlet, a routine, source code, object code, a shared library/dynamic link library, or one or more instructions. These software modules may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the executable code is stored in persistent storage.

The term “processing” may include launching a mobile application wherein launching should be interpreted as placing the mobile application in an open state and performing simulations of actions typical of human interactions with the mobile application. For example, a mobile application, such as a social media application, may be processed such that the mobile application is opened and actions such as user authentication, selecting to view a profile, scrolling through a newsfeed, and selecting and activating a link from the newsfeed are performed.

The term “mobile application” should be construed as a logic, software, or electronically executable instructions comprising a module, the mobile application being downloadable and installable on a network device. A mobile application may be a software application that is specifically designed to run on an operating system for a network device. Additionally, a mobile application may provide a graphical user interface (GUI) for the user of the network device.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

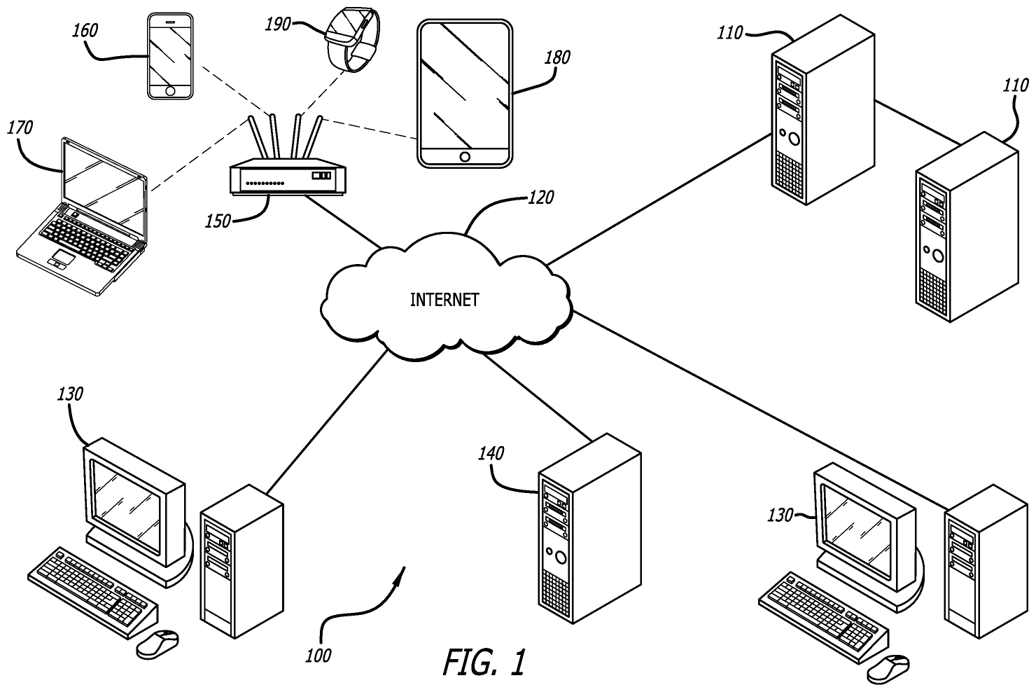

Referring to FIG. 1, a diagram of the interactive children's medication and wellness tracking system 100 in accordance with an embodiment of the invention is shown. The interactive children's medication and wellness tracking system 100 comprises a plurality of devices that are configured to transmit and receive data related to learning a variety of subjects. In many embodiments, children's medication servers 110 are connected to a network 120 such as, for example, the Internet. Children's medication servers 110 are configured to transmit a variety of data across the network 120 to any number of receiving devices such as, but not limited to, personal computers 130, and mobile computing devices including laptop computers 170, cellular phones 160, wearable computing devices 190, and portable tablet computers 180. In additional embodiments, many types of data related to the interactive children's medication and wellness tracking system 100 may be mirrored in additional cloud-based service provider servers 140. In certain embodiments, the data may be hosted on a cloud-based edge network system. In still additional embodiments, the children's medication servers 110 can be hosted as virtual servers within a cloud-based service.

In further embodiments, the sending and receiving of data can occur over the network 120 through wired and/or wireless connections. In the embodiment depicted in FIG. 1, the mobile computing devices 160, 170, 180, 190 are connected wirelessly to the network 120 via a wireless network access point 150. It should be understood by those skilled in the art that the types of wired and/or wireless connections between devices on the interactive children's medication and wellness tracking system 100 can be comprised of any combination of devices and connections as needed.

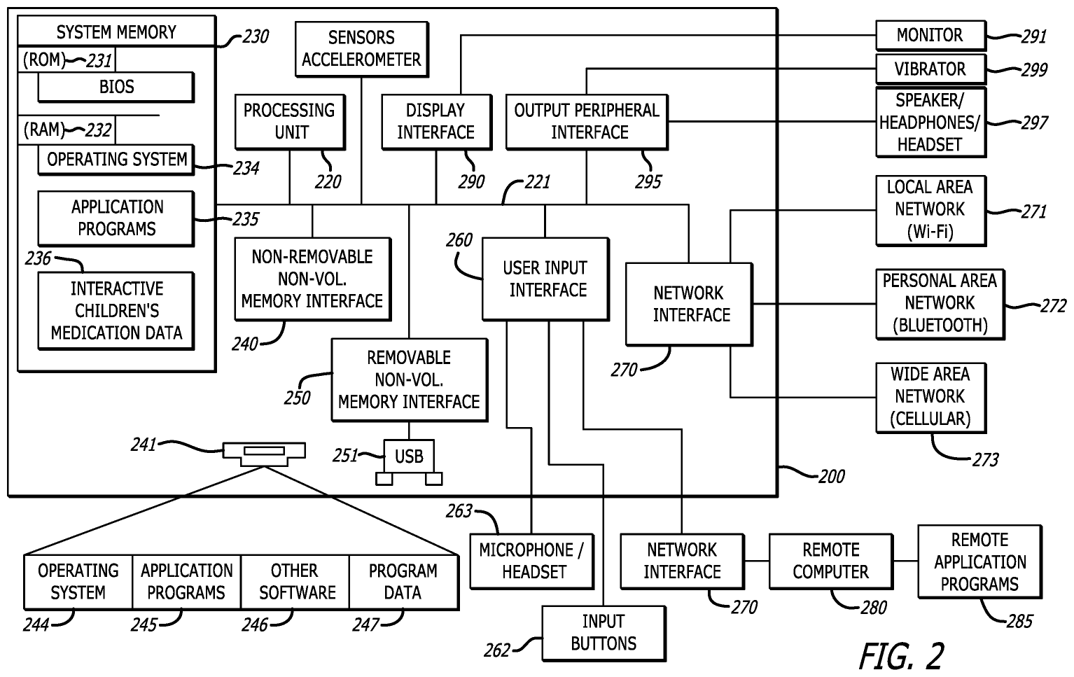

FIG. 2 is a schematic illustrating components of one or more interactive children's medication computing system 200 (also referred to as the “medication computing system 200”) in accordance with an embodiment of the invention is shown. One or more mobile computing devices, servers, cloud-based computers, edge hosting servers, or a combination thereof of the interactive children's medication and wellness tracking system 100 provided herein can be partially represented by the one or more components of the medication computing system 200 or wholly represented by all the components of the medication computing system 200.

Referring to FIG. 2, components of the medication computing system 200 can include, but are not limited to, a processing unit 220 having one or more processing cores, a system memory 230, and a system bus 221 that couples various system components including the system memory 230 to the processing unit 220. The system bus 221 can be any of several types of bus structures selected from a memory bus or memory controller, a peripheral bus, and a local bus using any of a variety of bus architectures.

The medication computing system 200 can include computing machine-readable media. The computing machine-readable media can be any available media that can be accessed by the medication computing system 200 and includes both volatile and non-volatile media, along with removable and non-removable media. By way of example and not limitation, computing machine-readable media use includes storage of information, such as computer-readable instructions, data structures, other executable software or other data. The computing machine-readable media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium that can be used to store the desired information and that can be accessed by the medication computing system 200. Transitory media such as wireless channels are not included in the computing machine-readable media. Communication media typically embody computer-readable instructions, data structures, other executable software, or other transport mechanisms and include any information delivery media. As an example, some medication computing systems 200 on a network might not have optical or magnetic storage.

The system memory 230 can include computing machine-readable media in the form of volatile and/or non-volatile memory such as read-only memory (ROM) 231 and random access memory (RAM) 232. A basic input/output system 233 (BIOS) containing basic routines configured for transferring information between elements within the medication computing system 200, such as during start-up, can be stored in the ROM 231. The RAM 232 can contain data and/or software immediately accessible to and/or presently being operated on by the processing unit 220. By way of example, and not limitation, FIG. 2 illustrates that the RAM 232 can include a portion of the operating system 234, the application programs 235, and interactive children's medication data 236.

The medication computing system 200 can also include other removable/non-removable volatile/nonvolatile computing machine-readable media. By way of example only, FIG. 2 illustrates a solid-state memory 241. Other removable/non-removable, volatile/nonvolatile computing machine-readable media that can be used in the example operating environment include, but are not limited to, USB drives and devices, flash memory cards, solid-state RAM, solid-state ROM, and the like. The solid-state memory 241 can be connected to the system bus 221 through a non-removable memory interface such as interface 240, and the USB drive 251 can be connected to the system bus 221 by a removable memory interface, such as interface 250.

The drives and their associated computing machine-readable media discussed above and illustrated in FIG. 2 provide storage of computer-readable instructions, data structures, other executable software and other data for the medication computing system 200. In FIG. 2, for example, the solid-state memory 241 is illustrated for storing operating system 244, application programs 245, other executable software 246, and program data 247. Note that these components can either be the same as or different from the operating system 234, the application programs 235, and interactive children's medication data 236. The operating system 244, the application programs 245, the other executable software 246, and the program data 247 are given different numbers here to illustrate that, at a minimum, that they can be different copies.

A user (e.g., a parent, guardian, caregiver, etc.) can enter commands and information into the medication computing system 200 through a user input interface 260 which can include input devices such as a keyboard, a touchscreen, software or hardware input buttons 262, a microphone 263, or a pointing device or scrolling input component such as a mouse, trackball, or touch pad. This input can be done directly on a medication computing system 200 or can be entered gathered from the Internet via social media databases and transmitted directly as input to the medication computing system 200.

Output generated from the medication computing system 200 can be provided to the user in a variety of ways. In many embodiments, the medication computing system 200 will include one or more display interfaces 290 which can be configured to format and drive output to a monitor 291. In additional embodiments, an output peripheral interface 295 can generate various signals to drive other accessories or devices to deliver output to the user. In the embodiment depicted in FIG. 2, the output peripheral interface 295 is communicatively connected to a speaker/headphones/headset output 297 and a vibrator 299 which can be configured to deliver a vibration to a user for example, to alert the user to newly received recall data.

The medication computing system 200 can operate in a networked environment using logical connections to one or more remote computers/client devices, such as a remote computing system 280. The remote computing system 280 can be a cloud-based server, a personal computer, a hand-held device, a router, a peer device or other common network node, and can include many or all of the elements described above relative to the medication computing system 200. The logical connections depicted in FIG. 2 can include a personal area network (“PAN”) 272 (e.g., Bluetooth®), a local area network (“LAN”) 271 (e.g., Wi-Fi), and a wide area network (“WAN”) 273 (e.g., cellular network), but the logical connections can also include other networks. Such networking environments can be found in offices, enterprise-wide computer networks, intranets and the Internet. A browser application can be resident on the computing device and stored in the memory.

When used in a LAN networking environment, the medication computing system 200 can be connected to the LAN 271 through a network interface or adapter 270, which can be, for example, a Wi-Fi adapter. When used in a WAN networking environment (e.g., Internet), the medication computing system 200 typically includes some means for establishing communications over the WAN 273 such as the network interface 270. With respect to mobile telecommunication technologies, for example, a radio interface, which can be internal or external, can be connected to the system bus 221 via the network interface 270, or some other appropriate mechanism. In a networked environment, other software depicted relative to the medication computing system 200, or portions thereof, can be stored in a remote memory storage device. By way of example, and not limitation, FIG. 2 illustrates remote application programs 285 as residing on a remote computing device 280. It will be appreciated that the network connections shown are examples and other means of establishing a communications link between the computing devices can be used.

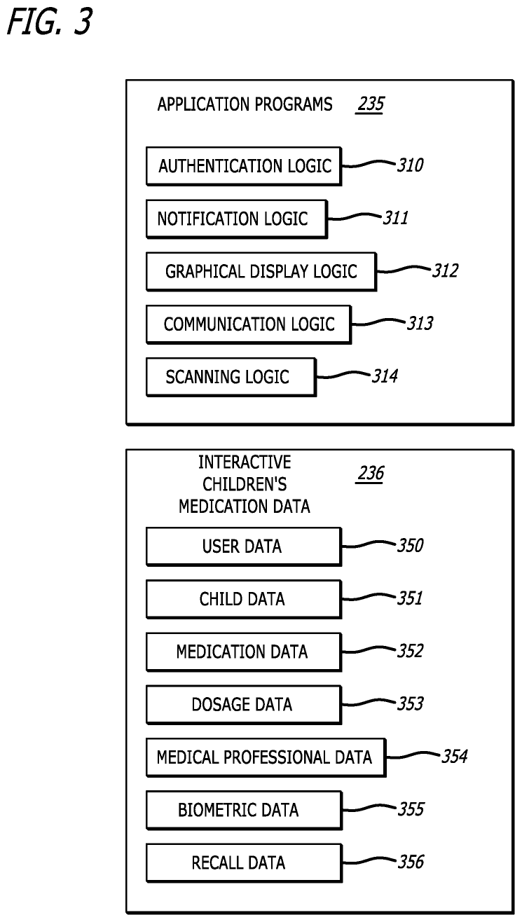

Referring to FIG. 3, a conceptual schematic illustrating various logics and data stores within an interactive children's medication computing device 200 in accordance with an embodiment of the invention is shown. As one skilled in the art will appreciate, the application programs 235 of the medication computing system 200 may comprise a plurality of specific applications or logics. In many embodiments, the application programs 235 may comprise authentication logic 310, notification logic 311, graphical display logic 312, communication logic 313, and/or scanning logic 314. It is contemplated that additional logics may be present in certain embodiments while other logics may be removed or collapsed within other logics as needed.

Likewise, the interactive children's medication data 236 can include a plurality of data types depending on the configuration and/or application of the medication computing system 200. In a variety of embodiments, the interactive children's medication data 236 may comprise user data 350, child data 351, medication data 352, dosage data 353, medical professional data 354, biometric data 355, and/or recall data 356. Similarly to the application programs 235, it is contemplated that additional data types may be present in certain embodiments while other data types may be removed or collapsed within other logics as needed.

In further embodiments, authentication logic 310 can be utilized by a processor to provide a log in system to limit access to the program to only authorized users. In certain cases, it may be desirable to limit access to the interactive children's medication application in order to avoid children changing data related to their medication and/or dosing. Additionally, it may useful to lock out access to only one parent such that multiple parents avoid giving their children excess doses of medicine. In some embodiments, the authentication logic can provide communication with an outside authentication system which may for example, allow a user to log into one or more social media sites to gather information related to their children and or medical history.

In additional embodiments, notification logic 311 can be utilized to generate and transmit a plurality of notifications related to the medical information available for at least one of the children being monitored by the system. In many embodiments, the notification logic 311 can generate a series of notifications that prompt a user to give a child another dose of medicine. These notifications can be automatically or dynamically generated. For example, the medication computing system 200 may provide the user with a prompt seeking to receive data associated with the approximate time correlating to the first dose of medicine given a child. The notification logic 311 may then evaluate the time needed to elapse for the next dose of medicine to be safely administered and generate a notification for transmission to the user to remind them to give the next dose of medicine approximately around the evaluated time.

In still further embodiments, notification logic 311 can be utilized to transmit other relevant data to a user. Such notifications may include relevant recall information associated with at least one medicine that was given to a child, availability of a medical professional to talk, vital statistics evaluated by biometric sensors on wearable devices 190 that exceed predetermined thresholds, and other message that can be relevant to the user.

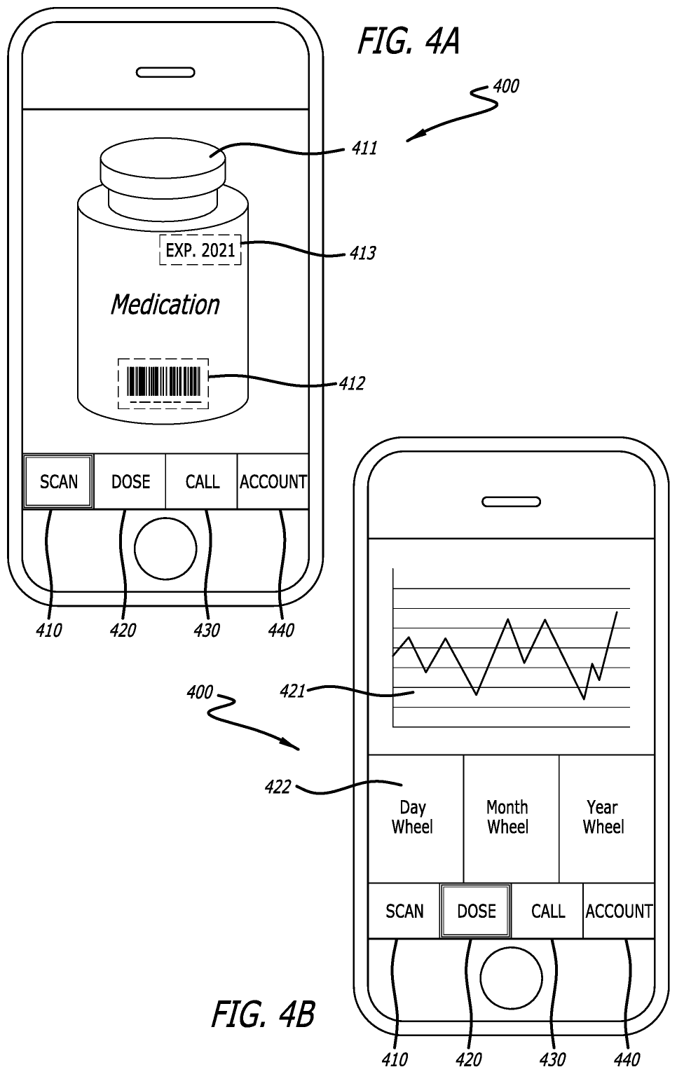

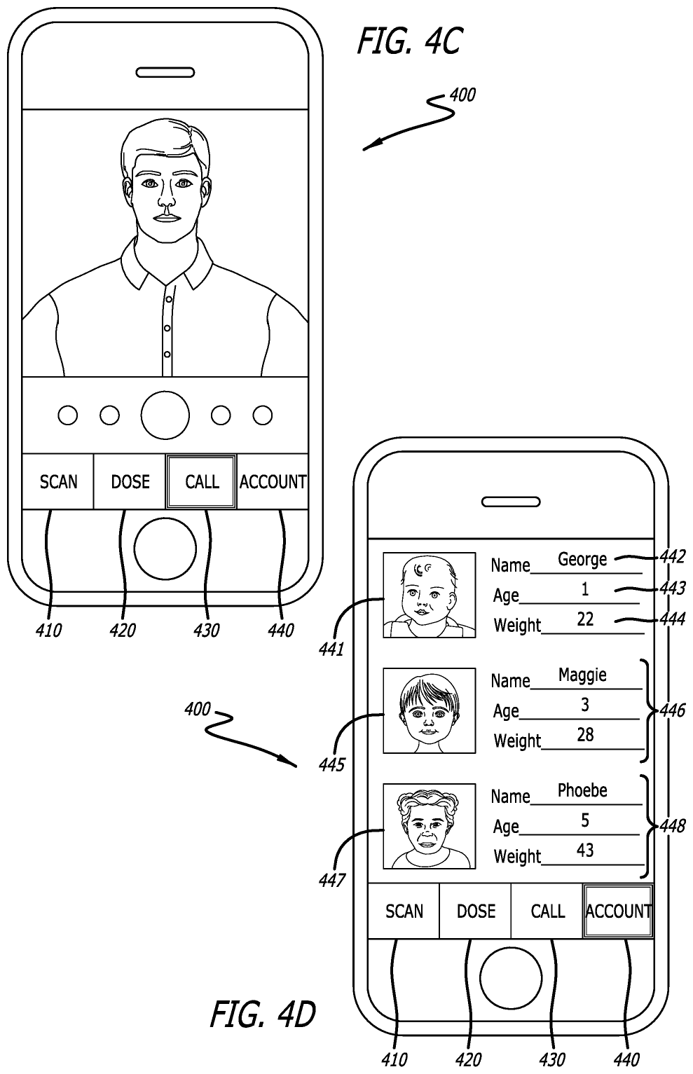

In more embodiments, graphical display logic 312 can generate a variety of graphical displays for users to receive and insert data related to the children's medical computing system. Examples of various graphical displays are shown in more detail below with reference to FIGS. 4A-4D. In certain embodiments, a portion of the graphical display processing can be offloaded to the operating system 234 of the medication computing system 200. Graphical display logic 312 can also be utilized to facilitate input from a user to generate user data 350 through a series of user interface prompts. In more embodiments, prompts generated by the graphical display logic 312 can be utilized to generate child data 351 which can be associated with at least one child of the user.

In still additional embodiments, communication logic 313 can be configured to facilitate communication between the medication computing system 200 and any remote servers and/or other computing devices. Some embodiments may utilize the communication logic 313 to establish and communicate with a children's medication server 110 to receive dosage data 353 in response to transmitting child data 351 and/or medication data 352. In further embodiments, communication logic 313 can facilitate receiving recall data 356 associated with at least one medication correlated with the medication data 352. In still further embodiments, communication logic 313 can facilitate the reception of biometric data from any of a plurality of biometric sensors which can be for example, found in a wearable device 190.

In more embodiments, scanning logic 314 can provide methods to scan various forms of data into the medication computing system 200 via at least one camera. In certain embodiments, a user can utilize the scanning logic to capture medication data 352 via barcode data. In additional embodiments, the scanning logic 314 may also be configured to capture expiration dates from medicine bottles and/or packages which can be added to the medication data 352. Those skilled in the art will appreciate that various methods including machine-learning algorithms may be utilized to gather medication data 352 and that certain methods may be able to determine medication based on optical character recognition or other patterns associated with various medicine packages.

In various embodiments, the interactive children's medication data 236 can include user data 350 which may correlate to various account and or personal information about the user of the application. By way of example and not limitation, the user may have login information and/or passwords stored within the user data 350. Other settings can also be stored within the user data 350 including notification settings, communication preferences (to outside devices, etc.), and/or display options.

In more embodiments, child data 351 comprises data associated with a plurality of children. Child data 351 may include, but is not limited to, age, weight, height, past medication history, past dosage history, medical professional contact information, emergency contact information, associated biometric and/or wearable device identifications, notification recipients, and/or photograph. As will be discussed in more detail below, the child data 351 is often utilized to determine various dosing levels of the medications assigned to them. Child data is often input by the user, but in certain embodiments may be captured from a network connection such as a social media account. In other embodiments, the child data 351 may be retrieved from a remote server that contains the necessary information. These embodiments may be utilized for example, with insurance companies who have the data easily available and/or schools when the interactive children's medication and wellness tracking system is being utilized by a school nurse or other medical professional user.

In many embodiments, medication data 352 corresponds to data related to any of the medications that can be assigned to a child. Medication data 352 may be input manually by a user, or it may be received from scanning logic 314 as the result of a scan of a medicine bottle/package. In certain embodiments, the medication data 352 may be received from a remote computing device such as a prescribing doctor's office or insurance company. The medication data 352 may comprise, but is not limited to, medication name, retail name, dosage, expiration date, side effects, prescribing doctor, source (doctor or over-the-counter), and/or number of doses within a package.

Similar to medication data 352, dosage data 353 can comprise various data related to the dosing of each medication. In a variety of embodiments, dosage data 353 may comprise number of doses prescribed/purchased, how many doses remain, amount of current dose, time of last dose taken, and/or level of success in providing dosing (being associated with amount of dose actually ingested by the child). Further embodiments may also have dosage data 353 comprise historical data related to each child's dosage amount, times, and/or completion for each medication. Aspects of dosage data 353 may be generated directly from input by a user. However, in many embodiments, the dosage data 353 can be dynamically generated based on a combination of stored child data 351, user input, medication data 352, and/or other dosage data 353 received from a remote children's medication server 140. In some embodiments, the dosage data 353 may be generated on the mobile application of the medication computing system 200. In other embodiments, the dosage data 353 may be generated on a remote computing device which transmits the dosage data 353 to the medication computing system 200.

In some embodiments, medical professional data 354 may be available that comprises contact information for various medical professionals. In additional embodiments, some medical professionals may be associated with the interactive children's medical tracking system 100 and can be available to answer questions. In these embodiments, a user may indicate via the graphical display logic 312 that they wish to speak with a medical professional whose contact information is stored as medical professional data 354. These medical professionals may already be known and established with the user prior to use or may be a listing of various available medical professionals who the user may search through to find a suitable person. An example of such communication is discussed in more detail in FIG. 4C below.

In certain embodiments, biometric data 355 can be gathered to further add functionality to the interactive children's medication and wellness tracking system 100. As those skilled in the art will appreciate, biometric data 355 can comprise any of a number of data associated with various vital statistics. These may include, but are not limited to, heart rate, blood pressure, temperature, wakefulness, activity level, blood sugar, and/or noise levels. A user may set any vital to have a particular threshold of acceptable ranges. Thus, with a predetermined upper and lower threshold in place, a user may for example, be notified once the biometric data 355 indicates the child has a temperature above a danger zone or below a fever break level.

In still further embodiments, the biometric data 355 may be associated with dangerous events including, but not limited to, falls, heart arrhythmia, and/or elevated blood sugar levels. In these embodiments, the user may be notified via a notification that the event is occurring and help may be required. In certain embodiments, the communication logic 313 may establish or generate a call to a rescue dispatch or other medical emergency provider once a particular second threshold has been reached past the initial notification threshold. In this way, the user may be notified to notice and intervene in response to a vital exceeding a first threshold, but outside emergency services may be automatically contacted in the event the biometric data 355 indicates breaking of the second threshold wherein the second threshold is set to a level that is more unsafe for a child compared to the first threshold.

In particular embodiments, recall data 356 can be generated that can be utilized to warn users of various recalls of medicines associated with the medication data 352. In many embodiments, recall data 356 can be generated by the communication logic 313 establishing a connection with an outside server which can provide recall data 356 for many medications. In certain embodiments, the medication computing system 200 may receive a large portion of recall data 356 and then sort and parse through to determine if any recall data 356 is relevant to medication associated with the medication data 352, while deleting/removing the rest. In one embodiment, the extraneous recall data 356 is retained and utilized to compare against newly scanned medicines, allowing a user to scan medicines in a store prior to purchase to see if any recall exists on the potential medication.

In other embodiments, the medication computing system 200 may ping and/or request specific recall data individually from the medication associated with the medication data 352. In this way, only data related to potential relevant recalls are transmitted to the medication computing system 200. Recall data 356 can be utilized to generate notifications from the notification logic 311 if the recall is labelled as urgent or has a high safety warning.

Referring to FIG. 4A, a conceptual mock-up of an interactive children's medication and wellness tracking application in scan mode 410 in accordance with an embodiment of the invention is shown. In many embodiments, the interactive children's medication and wellness tracking system 100 can include a mobile application operated on a mobile computing device 400. The mobile application may include multiple modes of operation. Although it should be understood that the interactive children's medication and wellness tracking system 100 can be configured to operate on a dedicated device, or with an application on a desktop or other computer. The embodiment depicted in FIG. 4A comprises a scan mode 410, dosage mode 420, call mode 430, and account management mode 440. As those skilled in the art will appreciate, the exact number, layout, and/or function of each mode may differ depending on the specific layout, features, and/or version of the mobile computing device 400.

The mobile application depicted in FIG. 4A has scan mode 410 selected which can utilize the camera located within the mobile computing device 400 to scan a medicine bottle 411. The scanning of the medicine bottle 411 can include scanning for a bar code 412 and/or other data including an expiration date 413 or packaging text (not shown). In further embodiments, the scanning may be done on one or more pills to determine type and currently given dosage, which can be verified as correct. In some embodiments, the scanning may be accomplished through methods provided by the operating system 234. In such embodiments, the scanning may utilize any of a variety of machine-learning algorithms to determine if relevant data resides within the frame of the mobile application camera window. Data captured from the scan mode 410 can be added to the medication data 352 which can be utilized to generate dosage data 353 and checked for recall data 356.

Referring to FIG. 4B, a conceptual mock-up of an interactive children's medication and wellness tracking application in dosage mode 420 in accordance with an embodiment of the invention is shown. When a user wishes to determine a proper dosage to provide, they may in many embodiments, enter the dosage mode 420 to enter in relevant data necessary to provide an accurate dosage calculation. In certain embodiments, the mobile application may provide the user a prompt area 422 to provide data related to the child including birthdate, and current weight. Input data may be displayed and shown how it relates to historical dosages in a graph view 421. In some embodiments, the graph view 421 may provide a means for showing the dosage of the child over time which can provide visual feedback to the parent that their current dosage is not out of the normal range of dosages they have given in the past. In other embodiments, the graph view 421 may be utilized to show data over time for a specific illness period to highlight vital statistics changing and/or dosages changing over time. It is contemplated that the graph view 421 and the prompt area 422 can comprise any particular type of graph and/or prompt and is not limited to the types of graphs and prompts depicted in FIG. 4B.

Referring to FIG. 4C, a conceptual mock-up of an interactive children's medication and wellness tracking application in call mode 430 in accordance with an embodiment of the invention is shown. In instances where medical professionals can be contacted, call mode 430 can be utilized to call or video chat. In certain embodiments, the medical professional may be contacted in call mode in direct response to a received second biometric data threshold being exceeded which can indicate a serious safety issue.

The medical professional may be shown as a static picture with a traditional voice call placed to them. In other embodiments, the professional may be available and shown as a video chat. In these instances, the medical professional may be able to receive video and interact with the child to make better medical decisions and judgments compared to standard telephonic connections. In further embodiments, the medical professional may be able to draw on screen or provide screen shots of relevant pictures or medical information which may aide in explaining to the user. The algorithms for audio and/or video chat may be utilized or provided by the operating system 434.

Referring to FIG. 4D, a conceptual mock-up of an interactive children's medication and wellness tracking application in account management mode 440 in accordance with an embodiment of the invention is shown. In the embodiment depicted in FIG. 4D, account management mode 440 includes inputs for adding and/or displaying the plurality of children associated with the mobile application. Each entry for a child can include a picture, 441, 445, 447 and vital statistics 442-444, 446, 448. For example, a first child has a first picture 441 with an entry for their name 442, age 443, and weight 444. The user may enter or change any of these items as needed. In certain embodiments, the user may be prompted to update the information if a certain amount of time has passed between the current mobile application usage and the previous usage. A second child can also include a picture 445 and vital statistics 446, along with a third child's picture 447, and vital statistics 448. As those skilled in the art will appreciate, any number of children can be displayed for use in the program depending on the application. Additionally, selecting each child may increase the viewable area associated with their data to display even more detail including further vital statistics, medical professional contacts, and/or medication history.

In more embodiments, the account management mode 440 can also include prompts to enter and/or update user data 350, medication data 352, medical professional data 354 and or settings related to wearable devices 190 and associated biometric data 355. As discussed above, further modes may be available depending on the level of functionality desired by either the user or the mobile application developer. Additionally, modes may be selected and or switched with means other than the selection buttons shown in FIGS. 4A-4D.

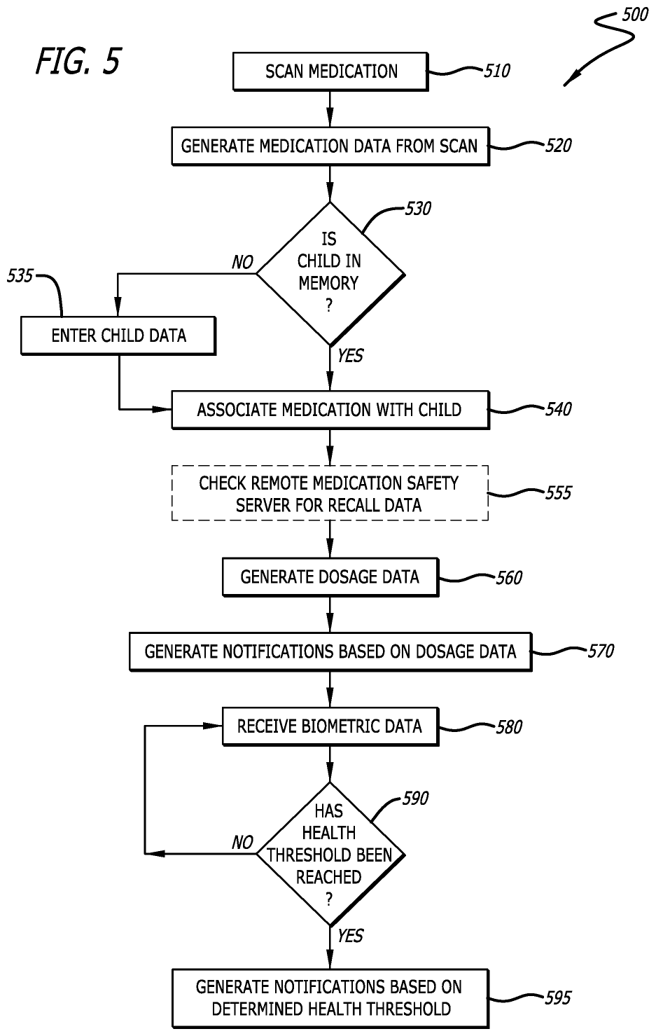

Referring to FIG. 5, a flowchart depicting an interactive children's medication and wellness tracking process 500 in accordance with an embodiment of the invention is shown. In many embodiments, the process 500 can begin with a medication being scanned by a user (block 510). The medication can be scanned via a camera or can be manually entered by the user if needed. In response to the scanned medication, the process 500 can generate medication data associated with the scanned medication (block 520). Descriptions of the types and detail of medication data is discussed above with reference to FIG. 3. The process 500 can check to see if child data is present that is associated with at least one child (block 530). In cases where no child data is found or a child is not found that is suitable for the medication scanned (e.g., only pre-teens child data is found when infant medications have been scanned), the user may be prompted to enter in additional child data (block 535).

The process 500 can prompt the user to verify and associate a scanned medication with a specific child within the child data (block 540). The scanned medication may be checked against recall data provided by a remote medication safety server to verify that no safety recalls exist on the medication (block 555). The checking of recall data is an optional step and can be accomplished at any stage after the scanning of medication within the process 500. In certain embodiments, the scanning for recall data may occur periodically or aperiodically after the process 500 has completed.

Once child data and medication data are obtained, the process 500 can generate dosage data (block 560). As shown above with more detail in the discussion of FIG. 3, the generation of the dosage data is typically done after a remote medication server has been contacted to verify current dosing guidelines for the specific medication scanned. The generation can occur remotely and be transferred back to the medication computing device, or general dosing guideline data may be transmitted to the medication computing device where the dosage data is generated within. As outlined above, the exact sequence and location of dosing data processing and generation can occur in various places and times based on the application desired.

In response to generating dosage data, the process 500 can generate a plurality of notifications based on the dosage data (block 570). The notifications can be automatically generated to occur every period of time after the initial dose is given to correspond when it is acceptable to administer another dose of medicine safely. In other cases, the notifications may be manually generated or approved by the user prior to use. The user may generate a predetermined notification plan that can be followed by the medication computing system when generating notifications. Notifications can be transmitted to a variety of devise including, but not limited to, mobile computing devices (smart phones, tablets, laptop computers, etc.), wearable devices, and/or medical devices.

Once administered, the process 500 can begin to receive biometric data associated with at least one of the children who has taken the medicine (block 580). The biometric data is often received from at least one wearable device 190 which can be worn by the at least one child. Alternatively, biometric data may be captured from devices that are not worn but utilized by the user to generate specific biometric data (e.g., blood pressure cuff, thermometer, scale, etc.). In various other embodiments, the biometric data may be imported from external sources. External sources may be, for example, from medical providers who have facilitated sharing of biometric data generated during a medical visit. The biometric data may also be manually inputted from one or more sources such as a caregiver who is monitoring the child. Each vital statistic captured by the biometric data can have a first threshold associated with it that can be set to generate a notification if the threshold is exceeded.

As a result, the process 500 verifies any of the first predetermined health-based thresholds has been reached (block 590). When no thresholds have been exceeded, the process 500 can return to listening for and/or receiving further biometric data (block 580). When the threshold has been exceeded, the process 500 can generate notifications as necessary based on the amount of excess the vital statistic is over the first pre-determined threshold or based on a previously existing notification policy (block 595). The thresholds to be monitored may be numerical in nature (e.g., temperature, heart rate, oxygenation levels, etc.) but may also include binary or non-numerical thresholds (e.g., ability to smell, skin color, movement levels, etc.)

As discussed above with more detail in the description of FIG. 3, notification logic may generate any acceptable notification in the form of push notifications, emails, texts, calls, audible alerts, vibrations, and/or direct messages. In certain embodiments, when the health threshold has been reached, generated notifications may be configured to facilitate one or more external actions. For example, a notification can be formatted to be transmitted to one or more medical devices that are themselves configured to receive external messages to start or stop an action. Finally, in some embodiments, the notifications can be configured to be sent to one or more caregivers or medical providers who are remotely located. In this way, a parent may be notified when a threshold has been met when they are away from the child/patient/etc.

Information as herein shown and described in detail is fully capable of attaining the above-described object of the present disclosure, the presently preferred embodiment of the present disclosure, and is, thus, representative of the subject matter that is broadly contemplated by the present disclosure. The scope of the present disclosure fully encompasses other embodiments that might become obvious to those skilled in the art, and is to be limited, accordingly, by nothing other than the appended claims. Any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for solutions to such problems to be encompassed by the present claims. Furthermore, no element, component, or method step in the present disclosure is intended to be dedicated to the public regardless of whether the element, component, or method step is explicitly recited in the claims. Various changes and modifications in form, material, work-piece, and fabrication material detail can be made, without departing from the spirit and scope of the present disclosure, as set forth in the appended claims, as might be apparent to those of ordinary skill in the art, are also encompassed by the present disclosure.

1. An interactive children's medication and wellness tracking system comprising:
a mobile application including:
a processor;
a camera;
a storage device commutatively coupled to the processor, the storage device comprises:
scanning logic configured to be processed by the processor to extract medication data from a picture captured by the camera;
graphical display logic to be processed by the processor to receive child data associated with at least one child;
communication logic to be processed by the processor to transmit the medication data to a children's medication server and to receive dosage data from the children's medication server in response to the transmitted medication data; and
wherein the graphical display logic further displays a proper dosage based on at least the child data, and dosage data.

2. The interactive children's medication and wellness tracking system of claim 1, wherein the calculation of the proper dosage is generated within the mobile application.

3. The interactive children's medication and wellness tracking system of claim 1, wherein the calculation of the proper dosage is generated within the children's medication server.

4. The interactive children's medication and wellness tracking system of claim 1, wherein the storage device further comprises notification logic to be processed by the processor to generate a plurality of notifications to the user associated with subsequent dosing times.

5. The interactive children's medication and wellness tracking system of claim 1, wherein the storage device further comprises notification logic to be processed by the processor to generate a plurality of notifications to the user associated with subsequent dosing amounts.

6. The interactive children's medication and wellness tracking system of claim 4, wherein the notifications are automatically generated subsequent to a prompt generated by the graphical user interface logic configured to determine the time a first dose of medicine was given.

7. The interactive children's medication and wellness tracking system of claim 5, wherein the communication logic is further configured to receive biometric data from at least one biometric device configured to gather biometric data from the at least one child.

8. The interactive children's medication and wellness tracking system of claim 7, wherein the dosing amounts generated for the plurality of notifications is generated in response to received biometric data.

9. The interactive children's medication and wellness tracking system of claim 1, wherein the communication logic is further configured to receive recall data from the children's medication server in response to the transmitted medication data and wherein the recall data corresponds with at least one medication associated with the medication data.

10. The interactive children's medication and wellness tracking system of claim 7, wherein the notification logic, in response to received biometric data, determines if at least one vital function associated with the biometric data exceeds a first pre-determined threshold and, in the event of the first threshold of the at least one vital function being exceeded, generates a plurality of notifications wherein the notifications are configured to alert a user to the current condition of the at least one vital function.

11. A method of tracking and providing interactive children's medication, comprising:
scanning medication with a camera to extract medication data from the captured pictures;
displaying a data input screen to a user configured to receive child data associated with at least one child;
receiving child data associated with the at least one child;
transmitting the medication data to a children's medication server;
receiving dosage data from the children's medication server in response to the transmitted medication data; and
generating a proper dosage for the medication based on at least the child data, and dosage data.

12. The method of claim 11, wherein the generation of the proper dosage is done within a mobile application.

13. The method of claim 11, wherein the generation of the proper dosage is done within a children's medication server.

14. The method of claim 11, wherein the method further comprises generating a plurality of notifications to the user associated with subsequent dosing times.

15. The method of claim 11, wherein the method further comprises generating a plurality of notifications to the user associated with subsequent dosing amounts.

16. The method of claim 14, wherein the notifications are automatically generated subsequent to a user prompt configured to determine the time a first dose of medicine was given.

17. The method of claim 15, wherein the method further comprises receiving biometric data from at least one biometric device configured to gather biometric data from the at least one child.

18. The method of claim 17, wherein the dosing amounts generated for the plurality of notifications is generated in response to received biometric data.

19. The method of claim 11, wherein the method further comprises receiving recall data from the children's medication server in response to the transmitted medication data and wherein the recall data corresponds with at least one medication associated with the medication data.

20. The method of claim 17, wherein the method further comprises, determining, in response to received biometric data, if at least one vital function associated with the biometric data exceeds a first pre-determined threshold and, in the event of the first threshold of the at least one vital function being exceeded, generates a plurality of notifications wherein the notifications are configured to alert a user to the current condition of the at least one vital function.

21. The method of claim 11, wherein child data is received from at least one social media database.
